(19) United States
(12) Patent Application Publication
(10) Pub. No.: US 2008/0207975 A1
(43) Pub. Date: Aug. 28, 2008

(54) Method For The Production Of Propene From Propane

(75) Inventors: Sven Crone, Limburgerhof (DE); Otto Machhammer, Mannheim (DE); Gotz-Peter Schindler, Mannheim (DE)

Correspondence Address: CONNOLLY BOVE LODGE & HUTZ, LLP, P O BOX 2207, WILMINGTON, DE, 19899, US

(73) Assignee: BASF AKTIENGESELLSCHAFT, Ludwigshafen (DE)

(21) Appl. No.: 11/722,367

(22) PCT Filed: Dec. 20, 2005
(86) PCT No.: PCT/EP2005/013700

§ 371 Date: Jun. 21, 2007

(30) Foreign Application Priority Data
Dec. 22, 2004 (DE) 10 2004 061 772.4

(51) Int. Cl. C07C 5/00

(52) U.S. Cl. 585/655

(57) ABSTRACT

The invention relates to a process for preparing propene from propane, comprising the steps:

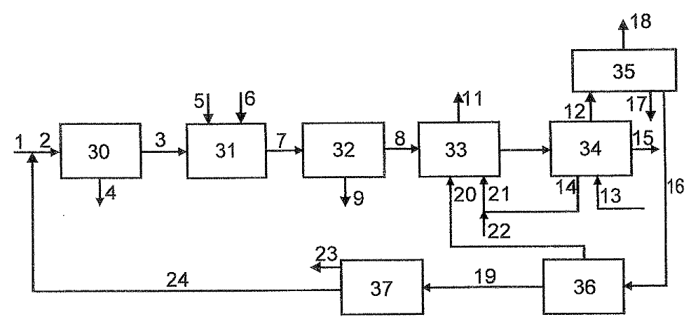

A) a feed gas stream a comprising propane is provided;

B) the feed gas stream a comprising propane and an oxygenous gas stream are fed into a dehydrogenation zone and propane is subjected to a nonoxidative catalytic, autothermal dehydrogenation to propene to obtain a product gas stream b comprising propane, propene, methane, ethane, ethene, nitrogen, carbon monoxide, carbon dioxide, steam and hydrogen;

C) product gas stream b is cooled and steam is removed by condensation to obtain a steam-depleted product gas stream c;

D) uncondensable or low-boiling gas constituents are removed by contacting product gas stream c with an inert absorbent and subsequently desorbing the gases dissolved in the inert absorbent to obtain a C₃ hydrocarbon stream d1 and an offgas stream d2 comprising methane, ethane, ethene, nitrogen, carbon monoxide, carbon dioxide and hydrogen;

E) the C₃ hydrocarbon stream d1 is cooled and compressed to obtain a liquid C₃ hydrocarbon stream e1;

F) the liquid C₃₋ hydrocarbon stream e1 is fed into a first distillation zone and separated distillatively into a stream f1 composed of propane and propene and a stream f2 comprising ethane and ethene;

G) stream f1 is fed into a (second) distillation zone and separated distillatively into a product stream g1 composed of propene and a stream g2 composed of propane, and stream g2 is recycled at least partly into the dehydrogenation zone.

The invention relates to a process for preparing propene from propane.

Propene is obtained on the industrial scale by dehydrogenating propane.

In the process, known as the UOP-oleflex process, for dehydrogenating propane to propene, a feed gas stream comprising propane is preheated to 600-700° C. and dehydrogenated in a moving bed dehydrogenation reactor over a catalyst which comprises platinum on alumina to obtain a product gas stream comprising predominantly propane, propene and hydrogen. In addition, low-boiling hydrocarbons formed by cracking (methane, ethane, ethene) and small amounts of high boilers (C₄⁺ hydrocarbons) are present in the product gas stream. The product gas mixture is cooled and compressed in a plurality of stages. Subsequently, the C₂ and C₃ hydrocarbons and the high boilers are removed from the hydrogen and methane formed in the dehydrogenation by condensation in a “cold box”. The liquid hydrocarbon condensate is subsequently separated by distillation by removing the C₂ hydrocarbons and remaining methane in a first column and separating the C₃ hydrocarbon stream into a propene fraction having high purity and a propane fraction which also comprises the C₄⁺ hydrocarbons in a second distillation column.

A disadvantage of this process is the loss of C₃ hydrocarbons by the condensation in the cold box. Owing to the large amounts of hydrogen formed in the dehydrogenation and as a consequence of the phase equilibrium, relatively large amounts of C₃ hydrocarbons are also discharged with the hydrogen/methane offgas stream unless condensation is effected at very low temperatures. Thus, it is necessary to work at temperatures of from −20 to −60° C. in order to limit the loss of C₃ hydrocarbons which are discharged with the hydrogen/methane offgas stream.

It is an object of the present invention to provide an improved process for dehydrogenating propane to propene.

The object is achieved by a process for preparing propene from propane, comprising the steps;

A) a feed gas stream a comprising propane is provided;

B) the feed gas stream a comprising propane and an oxygenous gas stream are fed into a dehydrogenation zone and propane is subjected to a nonoxidative catalytic, autothermal dehydrogenation to propene to obtain a product gas stream b comprising propane, propene, methane, ethane, ethene, nitrogen, carbon monoxide, carbon dioxide, steam and hydrogen;

C) product gas stream b is cooled and steam is removed by condensation to obtain a steam-depleted product gas stream c;

D) uncondensable or low-boiling gas constituents are removed by contacting product gas stream c with an inert absorbent and subsequently desorbing the gases dissolved in the inert absorbent to obtain a C₃ hydrocarbon stream d1 and an offgas stream d2 comprising methane, ethane, ethene, nitrogen, carbon monoxide, carbon dioxide and hydrogen;

E) the C₃ hydrocarbon stream d1 is cooled and, if appropriate, compressed to obtain a gaseous or liquid C₃ hydrocarbon stream e1;

F) the C₃ hydrocarbon stream e1 is, if appropriate, fed into a first distillation zone and separated distillatively into a stream f1 composed of propane and propene and a stream f2 comprising ethane and ethene;

G) stream e1 or f1 is fed into a (second) distillation zone and separated distillatively into a product stream g1 composed of propene and a stream g2 composed of propane, and stream g2 is recycled at least partly into the dehydrogenation zone.

In a first process part, A, a feed gas stream a comprising propane is provided. This generally comprises at least 80% by volume of propane, preferably 90% by volume of propane. In addition, the propane-containing feed gas stream A generally also comprises butanes (n-butane, isobutane). Typical compositions of the propane-containing feed gas stream are disclosed in DE-A 102 46 119 and DE-A 102 45 585. Typically, the propane-containing feed gas stream a is obtained from liquid petroleum gas (LPG). The propane-containing feed gas stream may be subjected to a purifying distillation to remove the butanes, in which a feed gas stream a having a very high propane content (>95% by volume) is obtained.

In one process part, B, the feed gas stream comprising propane is fed into a dehydrogenation zone and subjected to a nonoxidative catalytic dehydrogenation. In this process part, propane is dehydrogenated partially in a dehydrogenation reactor over a dehydrogenation-active catalyst to give propene. In addition, hydrogen and small amounts of methane, ethane, ethene and C₄⁺ hydrocarbons (n-butane, isobutane, butenes, butadiene) are obtained. Also obtained in the product gas mixture of the nonoxidative catalytic, autothermal propane dehydrogenation are carbon oxides (CO, CO₂), in particular CO₂, water and inert gases to a small degree. Inert gases (nitrogen) are introduced with the oxygen stream used in the autothermal dehydrogenation. In addition, unconverted propane is present in the product gas mixture.

The nonoxidative catalytic propane dehydrogenation is carried out autothermally. To this end, a gas comprising oxygen is additionally admixed with the reaction gas mixture of the propane dehydrogenation in at least one reaction zone and the hydrogen and/or hydrocarbon present in the reaction gas mixture is at least partly combusted, which directly generates in the reaction gas mixture at least some of the heat required for dehydrogenation in the at least one reaction zone. The gas comprising oxygen which is used is air or oxygen-enriched air having an oxygen content up to 70% by volume, preferably up to 50% by volume.

One feature of the nonoxidative method compared to an oxidative method is that free hydrogen is still present at the outlet of the dehydrogenation zone. In the oxidative dehydrogenation, free hydrogen is not formed.

The nonoxidative catalytic autothermal propane dehydrogenation may in principle be carried out in any reactor types known from the prior art. A comparatively comprehensive description of reactor types suitable in accordance with the invention is also contained in “Catalytica® Studies Division, Oxidative Dehydrogenation and Alternative Dehydrogenation Processes” (Study Number 4192 OD, 1993, 430 Ferguson Drive, Mountain View, Calif., 94043-5272, USA).

A suitable reactor form is the fixed bed tubular or tube bundle reactor. In these reactors, the catalyst (dehydrogenation catalyst and if appropriate a specialized oxidation catalyst) is disposed as a fixed bed in a reaction tube or in a bundle of reaction tubes. Customary reaction tube internal diameters are from about 10 to 15 cm. A typical dehydrogenation tube bundle reactor comprises from about 300 to 1000 reaction tubes. The internal temperature in the reaction tubes typically varies in the range from 300 to 1200° C., preferably in the range from 500 to 1000° C. The working pressure is customarily from 0.5 to 8 bar, frequently from 1 to 2 bar, when a low steam dilution is used, or else from 3 to 8 bar when a high steam dilution is used (corresponding to the steam active reforming process (STAR process) or the Linde process) for the dehydrogenation of propane or butane of Phillips Petroleum Co. Typical gas hourly space velocities (GHSV) are from 500 to 2000 h⁻¹, based on hydrocarbon used. The catalyst geometry may, for example, be spherical or cylindrical (hollow or solid).

The nonoxidative catalytic, autothermal propane dehydrogenation may also be carried out under heterogeneous catalysis in a fluidized bed, according to the Snamprogetti/Yarsintez-FBD process. Appropriately, two fluidized beds are operated in parallel, of which one is generally in the state of regeneration. The working pressure is typically from 1 to 2 bar, the dehydrogenation temperature generally from 550 to 500° C. The heat required for the dehydrogenation can be introduced into the reaction system by preheating the dehydrogenation catalyst to the reaction temperature. The admixing of a cofeed comprising oxygen allows the preheater to be dispensed with and the required heat to be generated directly in the reactor system by combustion of hydrogen and/or hydrocarbons in the presence of oxygen. If appropriate, a cofeed comprising hydrogen may additionally be admixed.

The nonoxidative catalytic, autothermal propane dehydrogenation is preferably carried out in a tray reactor. This reactor comprises one or more successive catalyst beds. The number of catalyst beds may be from 1 to 20, advantageously from 1 to 6, preferably from 1 to 4 and in particular from 1 to 3. The catalyst beds are preferably flowed through radially or axially by the reaction gas. In general, such a tray reactor is operated using a fixed catalyst bed. In the simplest case, the fixed catalyst beds are disposed axially in a shaft furnace reactor or in the annular gaps of concentric cylindrical grids. A shaft furnace reactor corresponds to a tray reactor with only one tray. The performance of the dehydrogenation in a single shaft furnace reactor corresponds to one embodiment. In a further, preferred embodiment, the dehydrogenation is carried out in a tray reactor having 3 catalyst beds.

In general, the amount of the oxygenous gas added to the reaction gas mixture is selected in such a way that the amount of heat required for the dehydrogenation of the propane is generated by the combustion of the hydrogen present in the reaction gas mixture and of any hydrocarbons present in the reaction gas mixture and/or of carbon present in the form of coke. In general, the total amount of oxygen supplied, based on the total amount of propane, is from 0.001 to 0.5 mol/mol, preferably from 0.005 to 0.25 mol/mol, more preferably from 0.05 to 0.25 mol/mol. Oxygen is used in the form of oxygenous gas which comprises inert gases, for example air or air enriched with oxygen.

The hydrogen combusted to generate heat is the hydrogen formed in the catalytic propane dehydrogenation and also any hydrogen additionally added to the reaction gas mixture as hydrogenous gas. The amount of hydrogen present should preferably be such that the molar H₂/O₂ ratio in the reaction gas mixture immediately after the oxygenous gas is fed in is from 1 to 10 mol/mol, preferably from 2 to 5 mol/mol. In multistage reactors, this applies to every intermediate feed of oxygenous and any hydrogenous gas.

The hydrogen is combusted catalytically. The dehydrogenation catalyst used generally catalyzes both the combustion of the hydrocarbons and of hydrogen with oxygen, so that in principle no specialized oxidation catalyst is required apart from it. In one embodiment, operation is effected in the presence of one or more oxidation catalysts which selectively catalyze the combustion of hydrogen to oxygen to water in the presence of hydrocarbons. The combustion of these hydrocarbons with oxygen to give CO, CO₂ and water therefore proceeds only to a minor extent. The dehydrogenation catalyst and the oxidation catalyst are preferably present in different reaction zones.

When the reaction is carried out in more than one stage, the oxidation catalyst may be present only in one, in more than one or in all reaction zones.

Preference is given to disposing the catalyst which selectively catalyzes the oxidation of hydrogen at the points where there are higher partial oxygen pressures than at other points in the reactor, in particular near the feed point for the oxygenous gas. The oxygenous gas and/or hydrogenous gas may be fed in at one or more points in the reactor.

In one embodiment of the process according to the invention, there is intermediate feeding of oxygenous gas and, if appropriate, of hydrogenous gas upstream of each tray of a tray reactor. In a further embodiment of the process according to the invention, oxygenous gas and, if appropriate, hydrogenous gas are fed in upstream of each tray except the first tray. In one embodiment, a layer of a specialized oxidation catalyst is present downstream of every feed point, followed by a layer of the dehydrogenation catalyst. In a further embodiment, no specialized oxidation catalyst is present. The dehydrogenation temperature is generally from 400 to 1100° C.; the pressure in the last catalyst bed of the tray reactor is generally from 0.2 to 5 bar, preferably from 1 to 3 bar. The GHSV is generally from 500 to 2000 h⁻¹, and, in high-load operation, even up to 1 000 000 h⁻¹, preferably from 4000 to 16 000 h⁻¹.

A preferred catalyst which selectively catalyzes the combustion of hydrogen comprises oxides and/or phosphates selected from the group consisting of the oxides and/or phosphates of germanium, tin, lead, arsenic, antimony and bismuth. A further preferred catalyst which catalyzes the combustion of hydrogen comprises a noble metal of transition group VIII and/or I of the periodic table.

The dehydrogenation catalysts used generally have a support and an active composition. The support generally consists of a heat-resistant oxide or mixed oxide. The dehydrogenation catalysts preferably comprise a metal oxide which is selected from the group consisting of zirconium dioxide, zinc oxide, aluminum oxide, silicon dioxide, titanium dioxide, magnesium oxide, lanthanum oxide, cerium oxide and mixtures thereof, as a support. The mixtures may be physical mixtures or else chemical mixed phases such as magnesium aluminum oxide or zinc aluminum oxide mixed oxides. Preferred supports are zirconium dioxide and/or silicon dioxide, and particular preference is given to mixtures of zirconium dioxide and silicon dioxide.

The active composition of the dehydrogenation catalysts generally comprises one or more elements of transition group VIII of the periodic table, preferably platinum and/or palladium, more preferably platinum. Furthermore, the dehydrogenation catalysts may comprise one or more elements of main group I and/or II of the periodic table, preferably potassium and/or cesium. The dehydrogenation catalysts may further comprise one or more elements of transition group III of the periodic table including the lanthanides and actinides, preferably lanthanum and/or cerium. Finally, the dehydrogenation catalysts may comprise one or more elements of main group III and/or IV of the periodic table, preferably one or more elements from the group consisting of boron, gallium, silicon, germanium, tin and lead, more preferably tin.

In a preferred embodiment, the dehydrogenation catalyst comprises at least one element of transition group VII, at least one element of main group I and/or II, at least one element of main group III and/or IV and at least one element of transition group III including the lanthanides and actinides.

For example, all dehydrogenation catalysts which are disclosed by WO 99/46039, U.S. Pat. No. 4,788,371, EP-A 705 136, WO 99/29420, U.S. Pat. No. 5,220,091, U.S. Pat. No. 5,430,220, U.S. Pat. No. 5,877,369, EP 0 117 146, DE-A 199 37 106, DE-A 199 37 105 and DE-A 199 37 107 may be used in accordance with the invention. Particularly preferred catalysts for the above-described variants of autothermal propane dehydrogenation are the catalysts according to examples 1, 2, 3 and 4 of DE-A 199 37 107.

Preference is given to carrying out the autothermal propane dehydrogenation in the presence of steam. The added steam serves as a heat carrier and supports the gasification of organic deposits on the catalysts, which counteracts carbonization of the catalysts and increases the onstream time of the catalysts. This converts the organic deposits to carbon monoxide, carbon dioxide and in some cases water.

The dehydrogenation catalyst may be regenerated in a manner known per se. For instance, steam may be added to the reaction gas mixture or a gas comprising oxygen may be passed from time to time over the catalyst bed at elevated temperature and the deposited carbon burnt off. The dilution with steam shifts the equilibrium toward the products of dehydrogenation. After the regeneration, the catalyst is reduced with a hydrogenous gas if appropriate.

In the autothermal propane dehydrogenation, a gas mixture is obtained which generally has the following composition: from 5 to 95% by volume of propane, from 1 to 40% by volume of propene, from 0 to 10% by volume of methane, ethane, ethene and C₄⁺ hydrocarbons, from 0 to 15% by volume of carbon dioxide, from 0 to 5% by volume of carbon monoxide, from 0 to 5% by volume of steam and from 0 to 30% by volume of hydrogen, and also from 1 to 50% by volume of inert gases (in particular nitrogen).

When it leaves the dehydrogenation zone, product gas stream b is generally under a pressure of from 1 to 5 bar, preferably from 1.5 to 3 bar, and has a temperature in the range from 400 to 700° C.

Product gas stream b may be separated into two substreams, in which case one substream is recycled into the autothermal dehydrogenation, corresponding to the cycle gas method described in DE-A 102 11 275 and DE-A 100 28 582.

In process part C, steam is initially removed from product gas stream b to obtain a steam-depleted product gas stream c. The removal of steam is carried out by condensation, by cooling and, if appropriate, compressing product gas stream b, and may be carried out in one or more cooling and, if appropriate, compression stages. In general, product gas stream b is cooled for this purpose to a temperature in the range from 0 to 80° C., preferably from 10 to 65° C. In addition, the product gas stream may be compressed, for example to a pressure in the range from 5 to 50 bar.

In one process part, D, the uncondensable or low-boiling gas constituents such as hydrogen, oxygen, carbon monoxide, carbon dioxide, nitrogen and a low-boiling hydrocarbon (methane, ethane, ethene) are removed from the C₃ hydrocarbons in an absorption/desorption cycle by means of a high-boiling absorbent to obtain a stream d1 which comprises the C₃ hydrocarbons and additionally also small amounts of ethene and ethane, and an offgas stream d2 which comprises the uncondensable or low-boiling gas constituents.

To this end, in an absorption stage, gas stream b is contacted with an inert absorbent to absorb C₃ hydrocarbons and also small amounts of the C₂ hydrocarbons in the inert absorbent and obtain an absorbent laden with C₃ hydrocarbons and an offgas d2 comprising the remaining gas constituents. Substantially, these are carbon oxides, hydrogen, inert gases and C₂ hydrocarbons and methane. In a desorption stage, the C₃ hydrocarbons are released again from the absorbent.

Inert absorbents used in the absorption stage are generally high-boiling nonpolar solvents in which the C₃ hydrocarbon mixture to be removed has a distinctly higher solubility than the remaining gas constituents to be removed. The absorption may be effected by simply passing stream c through the absorbent. However, it may also be effected in columns or in rotary absorbers. It is possible to work in cocurrent, countercurrent or crosscurrent. Suitable absorption columns are, for example, tray columns having bubble-cap trays, centrifugal trays and/or sieve trays, columns having structured packings, for example sheet metal packings having a specific surface area of from 100 to 1000 m²/m³ such as Mellapak® 250 Y, and columns having random packing. It is also possible to use trickle and spray towers, graphite block absorbers, surface absorbers such as thick-film and thin-film absorbers, and also rotary columns, pan scrubbers, cross-spray scrubbers, rotary scrubbers and bubble columns with and without internals.

Suitable absorbents are comparatively nonpolar organic solvents, for example aliphatic C₄-C₁₈-alkenes, naphtha or aromatic hydrocarbons such as the middle oil fractions from paraffin distillation, or ethers having bulky groups, or mixtures of these solvents, to which a polar solvent such as dimethyl 1,2-phthalate may be added. Suitable absorbents are also esters of benzoic acid and phthalic acid with straight-chain C₁-C₈-alkanols, such as n-butyl benzoate, methyl benzoate, ethyl benzoate, dimethyl phthalate, diethyl phthalate, and also heat carrier oils such as biphenyl and diphenyl ether, chlorine derivatives thereof, and triaryl alkenes. A suitable absorbent is a mixture of biphenyl and diphenyl ether, preferably in the azeotropic composition, for example the commercially available Diphyl®. Frequently, this solvent mixture comprises dimethyl phthalate in an amount of from 0.1 to 25% by weight. Suitable absorbents are also butanes, pentanes, hexanes, heptanes, octanes, nonanes, decanes, undecanes, dodecanes, tridecanes, tetradecanes, pentadecanes, hexadecanes, heptadecanes and octadecanes, or fractions which are obtained from refinery streams and comprise the linear alkenes mentioned as main components.

To desorb the C₃ hydrocarbons, the laden absorbent is heated and/or decompressed to a lower pressure. Alternatively, the desorption may also be effected by stripping, typically with steam, or in a combination of decompression, heating and stripping, in one or more process steps. For example, the desorption may be carried out in two stages, the second desorption stage being carried out at a lower pressure than the first desorption stage and the desorption gas of the first stage being recycled into the absorption stage. The absorbent regenerated in the desorption stage is recycled into the absorption stage.

In one process variant, the desorption step is carried out by decompressing and/or heating the laden absorbent. In a further process variant, stripping is effected additionally with steam.

The removal D is generally not entirely complete, so that, depending on the type of removal, small amounts or even just traces of the further gas constituents, in particular of the low-boiling hydrocarbons, may be present in the C₃ hydrocarbon stream d1.

To remove the hydrogen present in the offgas stream d2, the offgas stream may, if appropriate after cooling, for example in an indirect heat exchanger, be passed through a membrane, generally configured as a tube, which is permeable only to molecular hydrogen. The thus removed molecular hydrogen may, if required, be used at least partly in the dehydrogenation or else be sent to another utilization, for example to generate electrical energy in fuel cells. Alternatively, the offgas stream d2 may be incinerated.

In one process part, E, gas stream d1 is cooled, and it may additionally be compressed in one or more further compression stages. This affords a gaseous C₃ hydrocarbon stream e1 or a liquid condensate stream e1 composed of C₃ hydrocarbons. Stream e1 may comprise small amounts of C₂ hydrocarbons. In addition, an aqueous condensate stream e2 and, if appropriate, small amounts of an offgas stream e3 may be obtained. The aqueous condensate stream e2 is obtained generally when the dissolved gases are desorbed in step D by stripping with steam.

The compression may in turn be effected in one or more stages. In general, compression is effected overall from a pressure in the range of from 1 to 29 bar, preferably from 1 to 10 bar, to a pressure in the range of from 12 to 30 bar. Each compression stage is followed by a cooling stage in which the gas stream is cooled to a temperature in the range of from 15 to 80° C., preferably from 15 to 60° C. Subsequently, the compressed gas mixture is cooled to a temperature of from −10° C. to 60° C., preferably from −10° C. to 30° C. The liquid condensate streams e1 and e2 are separated from one another in a phase separation apparatus.

However, gas stream d1 may also only be cooled and fed in gaseous form to the first distillation zone, preferably when the desorption of the dissolved gases in process part D is brought about only by decompression and heating and not also by stripping with steam.

In one process part, F, the gaseous or liquid C₃ hydrocarbon stream e1 is fed into a first distillation zone and separated distillatively into a stream f1 comprising the C₃ hydrocarbons propane and propene and a stream f2 comprising the C₂ hydrocarbons ethane and ethene. To this end, the C₃ hydrocarbon stream e1 is generally fed into a C2/C3 separating column with typically from 20 to 80 theoretical plates, for example approx. 60 theoretical plates. This is operated generally at a pressure in the range of from 10 to 30 bar, for example at approx. 20 bar, and a reflux ratio of 2-30. The bottom temperature is generally from 40 to 100° C., for example approx. 60° C., the top temperature from −20 to 10° C., for example approx. 10° C.

A stream f1 composed of propane and propene is obtained at the bottom draw stream with an ethane/ethene content of generally <5000 ppm in total, preferably <1000 ppm, more preferably <500 ppm. Stream f2, which is preferably obtained at the top draw stream, may still comprise certain amounts of propane and propene and be recycled into the absorption stage for the removal thereof.

Process part F may also be dispensed with, especially when stream d1 or e1 has only a small proportion of C₂ hydrocarbons.

In a process part, G, the C₃ hydrocarbon stream e1 or f1 is fed into a second distillation zone and separated distillatively into a stream g1 comprising propene and a stream g2 comprising propane. To this end, the hydrocarbon stream f1 is generally fed into a C3 separating column (“C3 splitter”) having typically from 80 to 150 theoretical plates, for example approx. 100 theoretical plates. This is generally operated at a pressure in the range of from 10 to 30 bar, for example at approx. 20 bar, and a reflux ratio of 2-40. The bottom temperature is generally from 40 to 100° C., for example approx. 68° C., the top temperature from 30 to 60° C., for example approx. 60° C. Instead of a single C₃ separating column, it is also possible to use two C₃ separating columns, in which case the first column is operated at higher pressure, for example 25 bar, and the second column at lower pressure, for example 18 bar (2-column method). The top draw of the first column is liquefied in the bottom heater of the second column and the bottom draw of the first column is fed into the second column. Alternatively, a method with vapor compressors is also possible.

In a process part, H, stream g2 and a fresh propane stream may be fed into a third distillation zone in which a stream comprising C₄⁺ hydrocarbons is removed distillatively and the feed gas stream a having a very high propane content is obtained. The recycled stream g2 may be evaporated before entry into the third distillation zone. This can generate a coolant stream which can be used to cool at another point, for example for cooling at the top of the C2/C3 separating column.

The invention is illustrated in detail by the example which follows.

### EXAMPLE

The variant, shown in the figure, of the process according to the invention was simulated by calculation. The process parameters which follow were assumed.

A capacity of the plant of 369 kt/a of propene at running time 8000 h, corresponding to 46 072 kg/h of propene, is assumed.

In addition to 98% by weight of propane, the fresh propane stream c1 comprises approx. 2% by weight of butane. The fresh propane stream 1 is mixed with the propane recycle stream 24 from the C3 splitter 37 and fed to the C3/C4 separating column 30. In the C3/C4 separating column 30, which has 40 theoretical plates and is operated at operating pressure of 10 bar and a reflux ratio of 0.41, a high boiler stream 4 is removed and a propane stream 3 having a butane content of only 0.01% by weight is thus obtained. The propane stream 3 is preheated to 450° C., enters the dehydrogenation zone 31 and is subjected to an autothermal dehydrogenation. To this end, an oxygenous gas 6 and steam 5 are fed into the dehydrogenation zone 31. The conversion of the dehydrogenation is, based on propane, 50%, the selectivity of propene formation is 90%. In addition, 5% cracking products and 5% carbon oxides are formed by total combustion. The water concentration in the exit gas of the dehydrogenation zone is approx. 6% by weight, the residue oxygen content in the exit gas is 0% by weight, the exit temperature of the product gas mixture is 600° C. The product gas stream 7 is cooled and compressed in the compressor 32 starting from a pressure of 2.0 bar in 3 stages to a pressure of 15 bar. After the first and second compressor stage, cooling is effected in each case to 55° C. This provides an aqueous condensate 9 which is discharged from the process. The compressed and cooled gas stream 8 is contacted in the absorption column 33 with tetradecane 21 as an absorbent. The unabsorbed gases are drawn off as offgas stream 11 via the top of the column, the absorbent laden with the C₃ hydrocarbons is withdrawn via the bottom of the column and fed to the desorption column 34. In the desorption column 34, decompression to a pressure of 4 bar and stripping with high-pressure steam supplied as stream 13 desorbs the C₃ hydrocarbons to afford a stream 14 composed of regenerated absorbent and a stream 12 composed of C₃ hydrocarbons and steam. The regenerated absorbent 14 is supplemented with fresh absorbent 22 and recycled into the absorption column 33. At the top of the desorption column, the gas is cooled to 45° C., in the course of which further absorbent 14 condenses out. Also obtained is an aqueous phase which is removed in a phase separator and discharged from the process as stream 15. Subsequently, stream 12 is compressed in two stages to a pressure of 16 bar and cooled to a temperature of 40° C. This provides a small offgas stream 18, a wastewater stream 17 and a liquid C₃ hydrocarbon stream 16.

From the liquid C₃ hydrocarbon stream 16, a C₂ hydrocarbon stream 20 which additionally comprises certain amounts of C₃ hydrocarbons is removed via the top of a C2/C3 separating column 36 having 30 theoretical plates at 16 bar and a reflux ratio of 47. Stream 20 is recycled into the absorption column 33, where C₃ hydrocarbons present in stream 20 are removed. The bottom temperature in the C2/C3 separating column 36 is 41° C., the top temperature −5° C. The residue ethane content of the bottom draw stream 19 is 0.01% by weight. The bottom draw stream 19 is fed to a propane/propene separating column which has 120 theoretical plates and is operated at 16 bar with a reflux ratio of 21. The bottom temperature is 46° C., the top temperature 38° C. At the top, a propene stream 23 having a purity of 99.5% by weight of propene is obtained. The bottom draw stream 24 comprises approx. 98.5% by weight of propane and is recycled into the dehydrogenation zone 31.

TABLE

| Stream | 1 | 2 | 3 | 4 | 5 | 6 | 7 | 8 |
|---|---|---|---|---|---|---|---|---|
| Amount [kg/h] | 55060 | 109411 | 108301 | 1110 | 107 | 74475 | 182883 | 171749 |
| BUTANE | 0.0200 | 0.0101 | 0.0001 | 0.9887 | 0.0000 | 0.0000 | 0.0001 | 0.0001 |
| PROPANE | 0.9800 | 0.9824 | 0.9924 | 0.0100 | 0.0000 | 0.0000 | 0.2938 | 0.3129 |
| PROPENE | 0.0000 | 0.0075 | 0.0075 | 0.0000 | 0.0000 | 0.0000 | 0.2568 | 0.2734 |
| WATER | 0.0000 | 0.0000 | 0.0000 | 0.0000 | 1.0000 | 0.0000 | 0.0623 | 0.0016 |
| ETHENE | 0.0000 | 0.0000 | 0.0000 | 0.0000 | 0.0000 | 0.0000 | 0.0047 | 0.0050 |
| ETHANE | 0.0000 | 0.0000 | 0.0000 | 0.0000 | 0.0000 | 0.0000 | 0.0100 | 0.0106 |
| TDC | 0.0000 | 0.0000 | 0.0000 | 0.0013 | 0.0000 | 0.0000 | 0.0000 | 0.0000 |
| CO2 | 0.0000 | 0.0000 | 0.0000 | 0.0000 | 0.0000 | 0.0000 | 0.0293 | 0.0312 |
| H2 | 0.0000 | 0.0000 | 0.0000 | 0.0000 | 0.0000 | 0.0000 | 0.0079 | 0.0084 |
| O2 | 0.0000 | 0.0000 | 0.0000 | 0.0000 | 0.0000 | 0.2000 | 0.0000 | 0.0000 |
| N2 | 0.0000 | 0.0000 | 0.0000 | 0.0000 | 0.0000 | 0.8000 | 0.3258 | 0.3469 |
| CO | 0.0000 | 0.0000 | 0.0000 | 0.0000 | 0.0000 | 0.0000 | 0.0093 | 0.0099 |
| Temperature [° C.] | 20.0 | 30.0 | 26.9 | 78.2 | 350.0 | 600.0 | 600.0 | 30.0 |
| Pressure [bar] | 10.0 | 10.0 | 10.0 | 10.0 | 3.0 | 3.0 | 2.4 | 14.8 |

| Stream | 9 | 10 | 11 | 12 | 13 | 14 | 15 | 16 |
|---|---|---|---|---|---|---|---|---|
| Amount [kg/h] | 11133 | 902149 | 71238 | 112172 | 10000 | 800779 | 9021 | 101259 |
| BUTANE | 0.0001 | 0.0000 | 0.0000 | 0.0001 | 0.0000 | 0.0000 | 0.0002 | 0.0001 |
| PROPANE | 0.0001 | 0.0596 | 0.0001 | 0.4795 | 0.0000 | 0.0000 | 0.0002 | 0.5306 |
| PROPENE | 0.0003 | 0.0525 | 0.0039 | 0.4176 | 0.0000 | 0.0007 | 0.0003 | 0.4620 |
| WATER | 0.9979 | 0.0003 | 0.0030 | 0.0897 | 1.0000 | 0.0003 | 0.9976 | 0.0005 |
| ETHENE | 0.0000 | 0.0002 | 0.0120 | 0.0014 | 0.0000 | 0.0000 | 0.0000 | 0.0016 |
| ETHANE | 0.0016 | 0.0006 | 0.0252 | 0.0049 | 0.0000 | 0.0000 | 0.0008 | 0.0053 |
| TDC | 0.0000 | 0.8868 | 0.0000 | 0.0068 | 0.0000 | 0.9990 | 0.0009 | 0.0000 |
| CO2 | 0.0000 | 0.0000 | 0.0753 | 0.0000 | 0.0000 | 0.0000 | 0.0000 | 0.0000 |
| H2 | 0.0000 | 0.0000 | 0.0202 | 0.0000 | 0.0000 | 0.0000 | 0.0000 | 0.0000 |
| O2 | 0.0000 | 0.0000 | 0.0000 | 0.0000 | 0.0000 | 0.0000 | 0.0000 | 0.0000 |
| N2 | 0.0000 | 0.0000 | 0.8384 | 0.0000 | 0.0000 | 0.0000 | 0.0000 | 0.0000 |
| CO | 0.0000 | 0.0000 | 0.0240 | 0.0000 | 0.0000 | 0.0000 | 0.0000 | 0.0000 |
| Temperature [° C.] | 53.3 | 47.5 | 35.1 | 105.6 | 264.0 | 150.0 | 45.0 | 40.3 |
| Pressure [bar] | 14.8 | 14.8 | 14.8 | 4.0 | 50.0 | 4.0 | 3.9 | 16.1 |

| Stream | 17 | 18 | 19 | 20 | 21 | 22 | 23 | 24 |
|---|---|---|---|---|---|---|---|---|
| Amount [kg/h] | 1041 | 50 | 100423 | 836 | 800802 | 23 | 46072 | 54351 |
| BUTANE | 0.0004 | 0.0000 | 0.0001 | 0.0000 | 0.0000 | 0.0000 | 0.0000 | 0.0001 |
| PROPANE | 0.0020 | 0.4722 | 0.5347 | 0.0274 | 0.0000 | 0.0000 | 0.0038 | 0.9848 |
| PROPENE | 0.0020 | 0.4990 | 0.4646 | 0.1500 | 0.0007 | 0.0000 | 0.9950 | 0.0150 |
| WATER | 0.9806 | 0.0020 | 0.0005 | 0.0000 | 0.0003 | 0.0000 | 0.0010 | 0.0000 |
| ETHENE | 0.0000 | 0.0089 | 0.0000 | 0.1881 | 0.0000 | 0.0000 | 0.0000 | 0.0000 |
| ETHANE | 0.0045 | 0.0181 | 0.0001 | 0.6345 | 0.0000 | 0.0000 | 0.0002 | 0.0000 |
| TDC | 0.0105 | 0.0000 | 0.0000 | 0.0000 | 0.9990 | 1.0000 | 0.0000 | 0.0000 |
| CO2 | 0.0000 | 0.0000 | 0.0000 | 0.0000 | 0.0000 | 0.0000 | 0.0000 | 0.0000 |
| H2 | 0.0000 | 0.0000 | 0.0000 | 0.0000 | 0.0000 | 0.0000 | 0.0000 | 0.0000 |
| O2 | 0.0000 | 0.0000 | 0.0000 | 0.0000 | 0.0000 | 0.0000 | 0.0000 | 0.0000 |
| N2 | 0.0000 | 0.0000 | 0.0000 | 0.0000 | 0.0000 | 0.0000 | 0.0000 | 0.0000 |
| CO | 0.0000 | 0.0000 | 0.0000 | 0.0000 | 0.0000 | 0.0000 | 0.0000 | 0.0000 |
| Temperature [° C.] | 35.0 | 40.3 | 41.3 | −4.2 | 35.0 | 35.0 | 38.2 | 46.4 |
| Pressure [bar] | 16.1 | 16.1 | 16.0 | 16.0 | 14.8 | 14.8 | 15.9 | 15.9 |

1-9. (canceled)

10. A process for preparing propene from propane, comprising the steps:
A) providing a feed gas stream (a) comprising propane;
B) feeding said feed gas stream (a) and an oxygenous gas stream into a dehydrogenation zone and nonoxidatively catalytically, autothermally dehydrogenating propane to obtain a product gas stream (b) comprising propane, propene, methane, ethane, ethene, nitrogen, carbon monoxide, carbon dioxide, steam, and hydrogen;
C) cooling and removing the steam from said product gas stream (b) by condensation to obtain a steam-depleted product gas stream (c);
D) contacting said product gas stream (c) with an inert absorbent to remove uncondensable or low-boiling gas constituents and subsequently desorbing the gases dissolved in said inert absorbent to obtain a C₃ hydrocarbon stream (d1) and an offgas stream (d2) comprising methane, ethane, ethene, nitrogen, carbon monoxide, carbon dioxide, and hydrogen;
E) cooling and optionally compressing said C₃ hydrocarbon stream (d1) to obtain a gaseous or liquid C₃ hydrocarbon stream (e1);
F) optionally feeding said C₃ hydrocarbon stream (e1) into a first distillation zone and distillatively separating said C₃ hydrocarbon stream (e1) into a stream (f1) comprising propane and propene and a stream (f2) comprising ethane and ethene;
G) feeding stream (e1) or (f1) into a distillation zone and distillatively separating said stream (e1) or (f1) into a product stream (g1) comprising propene and a stream (g2) comprising propane and at least partly recycling stream (g2) into said dehydrogenation zone of step B).

11. The process according to claim 10, further comprising the additional step H) of feeding said stream (g2) and fresh propane into a third distillation zone and distillatively separating the resulting combination of said stream (g2) and fresh propane into said feed gas stream (a) and a stream comprising C₄⁺ hydrocarbons.

12. The process according to claim 10, wherein said product gas stream (b) is cooled in step C) to a temperature in the range of from 10° C. to 80° C.

13. The process according to claim 10, wherein said absorbent is selected from the group consisting of C₄ to C₁₈ alkanes, naphtha, and the middle oil fraction from paraffin distillation.

14. The process according to claim 10, wherein the gases dissolved in said absorbent are desorbed in step D) by stripping with steam.

15. The process according to claim 10, wherein said C₃ hydrocarbon stream (d1) is compressed in step E) to a pressure of from 5 to 25 bar.

16. The process according to claim 10, wherein said C₃ hydrocarbon stream (d1) is cooled to a temperature in the range from −10° C. to −60° C.

17. The process according to claim 10, wherein an aqueous condensate stream (e2) is additionally obtained in step F) and is removed in a phase separation apparatus from the liquid C₃ hydrocarbon stream.

18. The process according to claim 10, wherein said oxygenous stream of step B) is air or oxygen-enriched air with an oxygen content up to 70% by volume.
